(19) United States
(12) Patent Application Publication
(10) Pub. No.: US 2013/0116554 A1
(43) Pub. Date: May 9, 2013

(54) X-RAY IMAGING AT LOW CONTRAST AGENT CONCENTRATIONS AND/OR LOW DOSE RADIATION

(71) Applicants: Clemens Kaiser, Buckinghamshire (GB); Ben Newton, Buckinghamshire (GB)

(72) Inventors: Clemens Kaiser, Buckinghamshire (GB); Ben Newton, Buckinghamshire (GB)

(73) Assignee: GE HEALTHCARE AS, OSLO (NO)

(21) Appl. No.: 13/809,260

(22) PCT Filed: Jul. 12, 2011
(86) PCT No.: PCT/EP11/61843

§ 371 Date: Jan. 9, 2013

(30) Foreign Application Priority Data
Jul. 12, 2010 (NO) 20101001
Nov. 26, 2010 (NO) 20101660
Mar. 31, 2011 (NO) 20110499

(51) Int. Cl. A61K 49/04; A61B 6/03; A61M 5/00; A61B 6/00

(52) U.S. Cl. CPC A61K 49/0438; A61B 6/481; A61B 6/40; A61B 6/5258; A61B 6/035; A61M 5/007
USPC 600/425; 424/9.452; 600/431

(57) ABSTRACT

The present invention relates to X-ray examinations and to the improvement of patient safety during such. More specifically the invention relates to X-ray diagnostic compositions having ultra-low concentrations of iodine. The invention further relates to methods of X-ray examinations wherein a body is administered with an X-ray diagnostic composition and irradiated with a reduced radiation dose.

The present invention relates to X-ray examinations and to the improvement of patient safety during such. More specifically the invention relates to X-ray diagnostic compositions having ultra-low concentrations of iodine. The invention further relates to methods of X-ray examinations wherein a body is administered with an X-ray diagnostic composition and irradiated with a reduced radiation dose. In a particular embodiment, the invention relates to X-ray diagnostic compositions having ultra-low concentrations of iodine and to methods of X-ray examinations using such, wherein a body administered with the composition is irradiated with a reduced dose of x-ray radiation.

All diagnostic imaging is based on the achievement of different signal levels from different structures within the body so that these structures can be seen. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast or definition between a body structure or region of interest and its surroundings the higher the conspicuity or quality of the images and the greater their value to the physician performing the diagnosis. Moreover, the greater the contrast the smaller the body structures that may be visualized in the imaging procedures, i.e. increased contrast can lead to increased discernable spatial resolution and conspicuity.

For X-ray imaging, Computer Tomography (CT) provides a 3-dimensional spatial resolution and a contrast resolution that planar X-ray does not provide. Radiation dose varies considerably in radiology procedures. The average effective dose for some procedures are lower than 0.01 mSv (Table 1), whereas higher radiation doses are standard in CT procedures such as coronary angiography, where doses of 16 mSv or more are not uncommon, see (Table 2) from Mettler et al, Radiology, vol 248: 254-263 (2008).

Adult Effective Doses for Various Diagnostic Radiology Procedures

| | | Values |
|---|---|---|
| | Average | Reported in |
| | Effective | Literature |
| Examination | Dose (mSv) | (mSv) |
| Skull | 0.1 | 0.03-0.22 |
| Cervical spine | 0.2 | 0.07-0.3 |
| Th [symbol] racl [symbol] spine | 1.0 | 0.6-1.4 |
| Lumbar spine | 1.5 | 0.5-1.8 |
| Posteroanterior and lateral study of chest | 0.1 | 0.05-0.24 |
| Posteroanterior study of chest | 0.02 | 0.007-0.050 |
| Mammography | 0.4 | 0.10-0.60 |
| Abdomen | 0.7 | 0.04-1.1 |
| Pelvis | 0.6 | 0.2-1.2 |
| Hip | 0.7 | 0.18-2.71 |
| Shoulder | 0.01 | . . . |
| Knee | 0.005 | . . . |
| Other extremities | 0.001 | 0.002-0.1 |
| Dual x-ray absorp [symbol] metry (without CT) | 0.001 | 0.001-0.035 |
| Dual x-ray absorp [symbol] metry (with CT) | 0.04 | 0.003-0.06 |
| Intravenous urography | 3 | 0.7- [symbol] .7 |
| Upper gastrointestinal series | 6* | 1.5-12 |
| Small-bowel series | 5 | 3.0-7.8 |
| Barium enema | 8* | 2.0-18.0 |
| Endoscopic retrograde | 4.0 | . . . |
| cholangiopancreatography | | |

*Includes fluor [symbol] sc [symbol] py. [symbol]
indicates data missing or illegible when filed

Table 1 shows effective doses for various radiology procedures, Mettler et al, Radiology, vol 248: 254-263 (2008).

Adult Effective Doses for Various CT Procedures

| | Average | Values Reported |
|---|---|---|
| | Effective Dose | in Literature |
| Examination | (mSv) | (mSv) |
| Head | 2 | 0.9-4.0 |
| Neck | 3 | . . . |
| Chest | 7 | 4.0-18.0 |
| Chest for pulmonary embolism | 15 | 13-40 |
| Abdomen | 8 | 3.5-25 |
| Pelvis | 6 | 3.3-10 |
| Three-phase liver study | 15 | . . . |
| Spine | 6 | 1.5-10 |
| Coronary angiography | 16 | 5.0-32 |
| Calcium scoring | 3 | 1.0-12 |
| Virtual colonoscopy | 10 | 4.0-13.2 |

Table 2 shows effective doses for various CT procedures Mettler et al, Radiology, vol 248: 254-263 (2008).

The diagnostic quality of images is strongly dependent on the inherent noise level in the imaging procedure, and the ratio of the contrast level to the noise level or definition between contrast and noise can thus be seen to represent an effective diagnostic quality factor for diagnostic images. Achieving improvement in such a diagnostic quality factor has long been and still remains an important goal whilst keeping the patient safe, especially from excessive radiation. In techniques such as X-ray imaging one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade mark Gastrografen™), ionic dimers such as ioxaglate (marketed e.g. under the trade mark Hexabrix™), non-ionic monomers such as iohexyl (marketed e.g. under the trade mark Omnipaque™), iopamidol (marketed e.g. under the trade mark Isovue™), iomeprol (marketed e.g. under the trade mark Iomeron™) and the non-ionic dimer iodixanol (marketed under the trade mark Visipaque™).

The most widely used commercial non-ionic X-ray contrast agents such as those mentioned above are considered safe for clinical use. Contrast media containing iodinated contrast agents are used in more than 20 millions of X-ray examinations annually in the USA and the number of adverse reactions is considered acceptable. However, there is still a need for improved methods for X-ray, and CT images, providing high-quality images. This need is more apparent in patients/subjects with pre-existing diseases and conditions or immature/low renal function. This is because certain diseases and low renal function increase the chance of adverse reactions to injected iodinated contrast media. Pre-existing diseases of concern include lung disease, kidney disease, heart disease, liver disease, inflammatory disease, autoimmune disease and other comorbitities e.g. metabolic disorders (diabetes, hyperlipidaemia, hyperinsulinaemia, hypercholestraemia, hypertriglyceridaemia and hypertension), cardiovascular disease, peripheral vascular disease, atherosclerosis, stroke and congestive heart failure. Furthermore a subject's age is important since a greater number of adverse events are reported in the elderly, while immature renal function, as can be found in young children and infants, can also lead to prolonged circulation of contrast media and a greater number and intensity of adverse reactions.

The risk of adverse events is not limited to the effects of contrast media. Radiation associated with CT accounts for about 70-75% of the total ionizing radiation from diagnostic imaging. While these levels of radiation are well below those that cause deterministic effects (for example, cell death), there is concern that they may be associated with a risk of stochastic effects (such as cancer, cataracts and genetic effects). Those at greatest risk for developing radiation exposure-related cancer later in life are children and women in their 20s.

Approximately 33% of all paediatric CT examinations are performed in children in the first decade of life, with 17% in children at or under the age of 5 years. Exposure to radiation at an early age carries a risk because organs and tissues in children are more sensitive to the effects of radiation than those of an adult and they have a longer remaining life expectancy in which cancer may potentially form. In addition, the current prevalence of CT makes it more likely that children will receive a higher cumulative lifetime dose of medically related radiation than those who are currently adults.

Since such contrast media are conventionally used for diagnostic purposes rather than to achieve direct therapeutic effect, it is generally desirable to provide contrast media having as little as possible effect on the various biological mechanisms of the cells or the body as this will lead to lower toxicity and lower adverse clinical effect. The toxicity and adverse biological effects of iodinated contrast media are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the iodinated contrast agent structure and its physicochemistry, especially the osmolality of the contrast medium and the ionic composition or lack thereof of the contrast medium formulation. Desirable characteristics of an iodinated contrast agent have been considered to be low toxicity of the compound itself (chemotoxicity), low osmolality of the contrast medium, high hydrophilicity (solubility) and a high iodine content, frequently measured in mg iodine per ml of the formulated contrast medium for administration. The iodinated contrast agent must also be completely soluble in the formulation medium, usually an aqueous medium, and remain in solution during storage and administration.

The osmolalities of the commercial products, and in particular of the non-ionic compounds, is acceptable for most media containing dimers and non-ionic monomers although there is still room for improvement. In coronary angiography for example, injection into the circulatory system of a bolus dose of contrast medium may cause severe side effects. In this procedure, immediately after injection contrast medium rather than blood flows through the system for a short period of time, and differences in the chemical and physiochemical nature of the contrast medium and the blood that it replaces can cause undesirable adverse effects such as arrhythmias, QT prolongation, reduction in cardiac contractive force, reduction in oxygen carrying capacity of blood cells and tissue ischemia of the organ in which high levels of CM are present. Such effects are seen in particular with ionic contrast agents where chemotoxic and osmotoxic effects are associated with hypertonicity of the injected contrast medium. Contrast media that are isotonic or slightly hypotonic with the body fluids are particularly desired. Hypoosmolar contrast media have low renal toxicity which is particularly desirable.

In patients with acute renal failure, nephropathy induced by contrast medium remains one of the most clinically important complications of the use of iodinated contrast medium. Aspelin, P et al, The New England Journal of Medicine, Vol. 348:491-499 (2003) concluded that nephropathy induced by contrast medium may be less likely to develop in high risk patients when iodixanol, a hypoosmolar agent made isoosmolar with blood due to the addition of plasma electrolytes, is used rather than a low-osmolar, non-ionic contrast medium. These findings have later been reinforced by others, showing that Iodine contrast media osmolality is the key driver of contrast induced nephrotoxicity (CIN) and contrast media induced acute kidney injury.

The portion of the patient population considered as high-risk patients is increasing. To meet the need for continuous improvement of in vivo X-ray diagnostic agents for the entire patient population, there is a continuous drive in finding X-ray contrast agents and methods for x-ray imaging wherein the patient safety is optimized.

To keep the injection volume of the contrast media low it has been desirable to formulate contrast media with high concentration of iodine/ml, and still maintain the osmolality of the media at a low level, preferably below or close to isotonicity. This thinking corresponds well with the general rule that a higher iodine concentration is thought to provide a higher contrast enhancement. The development of non-ionic monomeric contrast agents and in particular non-ionic bis(triiodophenyl) dimers such as iodixanol (EP 108638) has provided contrast media with reduced osmotoxicity. This has allowed contrast with effective iodine concentration to be achieved with hypotonic solution, and has even allowed correction of ionic imbalance by inclusion of plasma ions while still maintaining the contrast medium at the desired osmolality (e.g. Visipaque™). However, to reduce the risk of adverse events, especially in susceptible subjects, to improve patient safety and to reduce costs, there is now a desire to reduce the amount of X-ray contrast media administered to patients undergoing X-ray examinations.

Yoshiharu Nakayama et al Radiology, 237: 945-951, 2005 is directed to methods of abdominal CT with low tube voltage, and concludes that by decreasing the tube voltage, the amount of contrast material can be reduced by at least 20% without image quality degradation. Further, it is reported that with a low tube voltage, the radiation dose can be reduced 57%.

Yoshiharu Nakayama et al AJR: 187, November 2006 is directed to methods of aortic CT angiography performed at a low tube voltage and reduced total dose of contrast material. In a first patient group 100 ml of iopamiron 300 mgl/ml is administered, while in a second group 40 ml of the same contrast media is administered. For the second group a 30% reduction in radiation dose is applied. The publication concludes that low-contrast and low-voltage scans are appropriate for lighter patients (<70 kg in body weight) with aortic disease. Moreover, this method is particularly valuable for follow-up studies of heavier patients (>70 kg) with renal dysfunction.

Kristina T. Flicek et al AJR, 195: 126-131, July 2010 is directed to the reduction of radiation dose for CT colonography (CTC) using adaptive statistical iterative reconstruction (ASIR) and suggests that the radiation dose during CTC can be reduced by 50% without significantly affecting the image quality when ASIR is used.

There is however still a desire to improve patient safety undergoing X-ray examinations, and particularly CT examinations, to reduce treatment costs and to make contrast-enhanced X-ray/CT available for patients previously referred to non-contrast-enhanced imaging.

The present invention provides a composition for, and a method of, X-ray imaging wherein the combination of reduced contrast media concentration and reduced X-ray radiation dose is applied to improve patient safety. This is a method to optimize patient safety, such as adult, child and infant patient safety, during X-ray/CT scanning procedures. There are five major variables to consider in the optimisation of images: radiation dose, contrast media concentration, contrast media dose, contrast media injection speed (rate), image quality and hitherto, three major variables to consider in the optimization of patient safety and the minimization of patient risk. These are the radiation dose, the contrast media dose and the image quality. The applicant has tested and surprisingly found that contrast media concentration can be reduced to unexpectedly low levels without compromising the contrast to noise and/or quality of the obtained X-ray images.

By the compositions and methods of the invention, there are several objectives achieved. Considerable cost savings can be made by the reduction of costs by reducing use of higher concentration contrast media as to achieve Cost of Goods and raw material savings. Further, there are indirect cost savings associated with the reduction of radiation so in total there may be a reduced treatment cost. Most importantly there are patient safety benefits through the combination of reduced iodine concentration and total dose of contrast media and reduced radiation exposure. The lower radiation dose of X-ray/CT procedures is especially beneficial for paediatric (child and infant) X-ray/CT and in those high risk patients with pre-existing disease where single or repeated contrast enhanced X-ray and CT scans are needed to diagnose the status, development or indeed reduction of disease in response to physician intervention. The lower iodine concentration exposure is especially beneficial to patients with pre-existing disease, such as reduced heart and kidney function. Thus the preserved or higher quality images are achieved and adverse events should be minimized. Images of sufficient quality can be obtained at low radiation doses for more patients, typically for those who were not previously referred for contrast enhanced scans, patients who require repeated scans, e.g. to aid therapeutic monitoring or disease management, or patients with risk factors e.g. due to radiation exposure or patient risk factors. Wth the composition and method of the invention an optimum balance regarding image quality, radiation and iodine concentration per individual patient can be achieved by either lowering iodine concentration and/or by lowering radiation dose.

Hence, in a first aspect the invention provides an X-ray diagnostic composition comprising an iodinated X-ray contrast agent together with a pharmaceutically acceptable carrier or excipient, wherein the composition has an ultra-low concentration of iodine. In one embodiment, the composition comprises a mixture of two or more iodinated X-ray contrast agents.

“Contrast agents”, are agents that comprise a material that can significantly attenuate incident X-ray radiation causing a reduction of the radiation transmitted through the volume of interest. After undergoing CT image reconstruction and typical post-processing, this increased X-ray attenuation is interpreted as an increase in the density of the volume or region of interest, which creates a contrast enhancement or improved definition in the volume comprising the contrast agent relative to the background tissue in the image.

The terms composition, X-ray diagnostic composition and contrast media will be used interchangeably in this document and have the same meaning.

By the term “ultra-low concentration” (ULC) of iodine we define the concentration to be 10-170 mgl/ml, or more preferably 10-150 mgl/ml, even more preferably 10-100 mgl/ml, and most preferably 10-75 mgl/ml. In a particularly preferred embodiment the iodine concentration is less than 100 mgl/ml. The concentration of the X-ray composition has been found to be important as the composition, when administered to a body, replaces blood. By lowering the radiation dose of the X-ray tube i.e by lowering tube voltage (kilo volt peak or kVp), i.e. the difference in potential between the cathode and anode, and administering ultra-low concentrations of iodine, the image quality, i.e. the contrast effect, is actually maintained or improved. This is because the attenuation value of iodinated enhancements is increased at a lower tube voltage as the dose of radiation has an average energy spectrum substantially corresponding to the k-edge of iodine, resulting in higher enhancement. Iodine HU values (Hounsfield Units) in the CT image are greater, i.e. the image quality is improved, at lower kVps because the average energy of the spectrum is closer to the k-edge of iodine (33.2 keV (kilo electron volts)) thus the increased attenuation coefficient of iodine at lower x-ray energies results in higher CT image HU values.

To clarify, it is the actual concentration of the material, preferably iodine, that attenuate incident X-ray radiation, that is lowered, and not only the dose of iodinated contrast media (volume). As a consequence, if the volumes of injected iodinated contrast agent remain the same and the concentration of iodine based contrast agent is reduced, the total amount of injected iodinated contrast agent into the body will be reduced. Using the composition of the invention comprising ultra-low concentrations of iodine, or using the method of the second aspect, has benefits over just reducing the overall standard dose of diagnostic composition or reducing the rate of administration of this. The concentration of iodine has been found to be more important than the dose for image ability since the contrast media pushes the blood out of the way and i.e. displaces or replaces blood, so that it alone is “imaged”. Since the overall contrast media dose is reduced because the contrast media concentration is reduced the dose of contrast agent is important for patient safety.

The contrast agent of the claimed composition is in one embodiment an iodinated X-ray compound. Preferably, the composition of the invention is a low-osmolar contrast media (LOCM). Preferably the contrast agent is a non-ionic iodinated monomeric compound or a non-ionic iodinated dimeric compound, i.e. a compound comprising single triiodinated phenyl groups or a compound comprising two linked triiodinated phenyl groups. However, trimeric, tetrameric and pentameric compounds are also included. This is because as the number of multimers increases the osmolality decreases. This is important because it means more serum electrolytes may be added to the solution to make it isotonic. Thus what is injected is mostly plasma electrolytes. In addition, since it is known that the viscosity increases with increasing numbers of multimers, the ULC approach may mean that multimeric agents are now acceptable for use since the low concentration required for imaging would lower the overall viscosity making it possible to practically use these compounds. Relevant monomeric and dimeric compounds are provided by the applicant's application WO2010/079201. Particularly relevant monomeric compounds are described in WO97/00240 and in particular the compound BP257 of example 2, and additionally the commercially available compounds iopamidol, iomeprol, ioversol, iopromide, ioversol, iobitridol, iopentol and iohexyl. Most particularly preferred are the compounds iopamidol and iohexyl.

Particularly relevant dimeric compounds are compounds of formula (I) of two linked triiodinated phenyl groups, denoted non-ionic dimeric compounds,

R—N(CHO)—X—N(R⁶)—R Formula (I)

and salts or optical active isomers thereof,
wherein
X denotes a C₃ to C₈ straight or branched alkylene moiety optionally with one or two CH₂ moieties replaced by oxygen atoms, sulphur atoms or NR⁴ groups and wherein the alkylene moiety optionally is substituted by up to six —OR⁴ groups;
R⁴ denotes a hydrogen atom or a C₁ to C₄ straight or branched alkyl group;
R⁶ denotes a hydrogen atom or an acyl function, such as a formyl group; and
each R independently is the same or different and denotes a triiodinated phenyl group, preferably a 2,4,6-triiodinated phenyl group, further substituted by two groups R⁵ wherein each R⁵ is the same or different and denotes a hydrogen atom or a non-ionic hydrophilic moiety, provided that at least one R⁵ group in the compound of formula (II) is a hydrophilic moiety. Preferred groups and compounds are outlined in applications WO2010/079201 and WO2009/008734 which are incorporated herein by reference.

Particularly preferred dimeric contrast agents that can be used in the composition or the method of the invention are the compounds iodixanol (Visipaque) and the compound of formula (II):

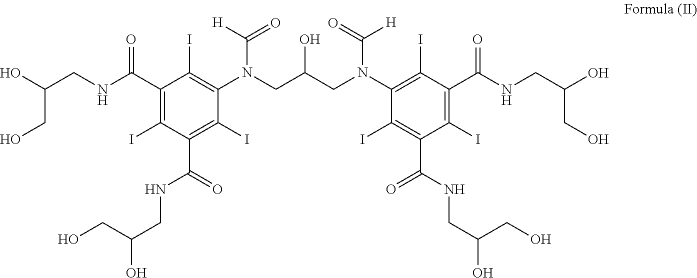 [SMILES: O=CN(CC(O)CN(C=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

The compound of formula (II) has been given the International Nonproprietary Name loforminol.

Hence in a preferred embodiment, the invention provides a composition comprising iodixanol or ioforminol, or both, wherein the composition has an ultra-low concentration of iodine.

The X-ray diagnostic composition of the invention may be in a ready to use concentration or may be a concentrate form for dilution prior to administration or it could be an amorphous powder that could be mixed with plasma electrolytes prior to administration. It may be desirable to make up the solution's tonicity by the addition of plasma cations so as to reduce the toxicity contribution that derives from the imbalance effects following bolus injection. In particular, addition of sodium, calcium and magnesium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used. It is possible to add electrolytes to the contrast medium to lower the cardiovascular effects. In one embodiment, the invention provides a composition dose, such as an x-ray diagnostic dose for administration, wherein the composition comprises an ultra-low concentration of iodine, and wherein the total volume of the composition is between 1 and 50 ml.

For X-ray diagnostic compositions which are administered by injection or infusion, the desired upper limit for the solution's viscosity at ambient temperature (20° C.) is about 30 mPas, however viscosities of up to 50 to 60 mPas and even more than 60 mPas can be tolerated. For X-ray diagnostic compositions given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O. With the composition of the invention such viscosity, osmolality and iodine concentrations targets can be met. Indeed, effective iodine concentrations can be reached with hypotonic solutions, i.e. with less than 200 mOsm/kg H₂O.

The X-ray diagnostic composition can be administered by injection or infusion, e.g. by intravascular administration. In one embodiment, the X-ray diagnostic composition is administered as a rapid intravascular injection, in another embodiment it is administered as a steady infusion. Alternatively, X-ray diagnostic composition may also be administered orally. For oral administration the composition may be in the form of a capsule, tablet or as liquid solution.

In a second aspect the invention provides a method of X-ray examination comprising

administering to a body an X-ray diagnostic composition comprising an x-ray contrast agent,
applying a reduced radiation dose to the body,
examining the body with a diagnostic device and
compiling data from the examination.

In one embodiment the only purpose of the method of the invention is to obtain information. The method may include analysing the data. In another embodiment, the method further includes a step of comparing the obtained information with other information so that a diagnosis can be made. In one embodiment, the method for examination is a method of diagnosis or is an aid for diagnosis. The reduced radiation dose is applied to the body, such as to a specific region of interest of the body.

Currently, X-ray/CT equipment algorithms only consider image quality and radiation dose as parameters when optimizing (i.e. lowering) radiation dose and/or improving image quality. Generally, the dose of radiation required to obtain a certain image quality in X-ray/CT scans can be reduced using advanced algorithms to reduce image noise associated with lower radiation exposure during the acquisition of images. In addition, applicant has now found that by decreasing the tube voltage, the amount of contrast material can be reduced to unexpectedly low levels by reducing the concentration without image quality degradation.

In cases where X-ray/CT scans require enhanced optimal images a contrast agent containing an attenuating material with high atomic number, e.g. iodine-containing contrast media is administered to improve contrast and allow for required image quality. Factors that impact the decision to use an X-ray diagnostic composition or not are patient risk factors such as body weight (obesity), low renal function, low liver function, age (infants, children and elderly) and/or comorbitities e.g. metabolic disorders (diabetes, hyperlipidaemia, hyperinsulinaemia, hypercholestraemia, hypertriglyceridaemia and hypertension), cardiovascular disease, peripheral vascular disease, atherosclerosis, stroke, congestive heart failure or type of procedure, e.g., intravenous, intraarterial, peripheral, cardiac, angiography and CT.

Although it has been shown that low dose contrast media and low-voltage scans are appropriate for lighter patients (<70 kg in body weight) with aortic disease (Nakayama et al 2006), the method of the present invention preferably includes the use of “ultra-low concentration iodine” compositions currently not considered or available in order to make the most of the reduction in radiation dose and kVp without compromising image quality and effective diagnosis. This method could also be applicable to material nanoparticles of high atomic number. It furthermore may include the use of advanced image reconstruction algorithms that are specifically designed to remove or reduce the soft-tissue noise resulting from the use of low radiation/low kVp scans in conjunction with the administration of ultra-low concentration of iodine. Hence, the optimization includes optimization of contrast media concentration and dose, in addition to radiation dose and image quality by effective reconstruction as parameters when determining optimum patient centric scan parameters.

In the state of the art there has been a trade-off between radiation dose and image quality. To achieve higher spatial resolution, higher radiation doses have been applied. Further, to have less noise, the radiation doses have been increased. At the same time there is a need to keep radiation doses down, e.g. due to the lifetime risk of developing cancer. By the method of the invention the radiation doses are low, without compromising the image quality because ultra-low concentrations of contrast media are administered. In one embodiment, the method includes administration of a composition comprising an ultra-low concentration of iodine, wherein the total volume of the composition is 1-50 ml.

Several techniques for achieving a reduction in the radiation dose during X-ray examinations, such as CT examinations, exist. One technique is to use low tube voltage. In one embodiment of this aspect, a polychromatic radiation spectrum is provided by tube voltages in the range of 70-150 kVp (kVp=kilo volt peak), such as 70-140 kVp, more preferably 70-120 kVp, even more preferably 70-85 kVp and most preferably 70-80 kVp. This will typically provide x-ray spectra of 30-140 keV (for 140 kVp tube voltage), more preferably 30-120 keV (for 120 kVp tube voltage), even more preferably 30-85 (for 85 kVp tube voltage) and most preferably 30-80 keV (for 80 kVp tube voltage). Hence, the tube voltage is most preferably below 80 kVp. Accordingly, when the body has been administered with the X-ray diagnostic composition, preferably with an ultra-low concentration of iodine, the x-ray/CT equipment is operated such that the body is irradiated with X-rays, preferably in accordance with CT, with a tube voltage as provided above. Today, the majority of abdominal CT scans are e.g. taken at 120 kVp. Wth the method of the invention, using an ultra-low concentration of iodine, this tube voltage, and accordingly the radiation dose, can be reduced as suggested without compromising on the image quality. Equivalent or better conspicuity, i.e. equal or higher contrast to noise ratio, of iodinated structures can be achieved when reducing the radiation dose, for instance from 140 kVp to 80 kVp or to values as low as 70 kVp. This is because the average energy of the polychromatic spectrum is closer to the k-edge of iodine (33.2 keV). The K-edge describes a sudden increase in the attenuation coefficient of X-ray photons just above the binding energy of the K shell electrons of the atoms interacting with the X-ray photons. The sudden increase in attenuation is due to photoelectric absorption/attenuation of the X-rays. Iodine has K shell binding energies for absorption/attenuation of X-rays of 33.2 keV, which is not necessarily close to the mean energy of most diagnostic X-ray beams. Thus, at lower photon energy more X-rays can be attenuated by iodine. Extrapolating such phenomena to contrast enhanced scanning procedures in the clinical setting, the use of low energy photons (i.e. low radiation), brighter images can be obtained. Alternatively, if less iodine is administered, equivalent image intensity could result. The balance between the low X-ray energy and the low amount (concentration of iodine) required to render images that are equivalent in quality and intensity as standard X-ray energy scans at normal or standard iodine concentrations, is of critical importance. Hence, in one embodiment of the method of the invention the dose of radiation applied has an average energy spectrum substantially corresponding to the k-edge of iodine.

Furthermore, if not properly addressed, the lowering of tube voltage and x-ray photon energy to reduce patient radiation dose and the resulting increase in iodine attenuation and image brightness could be the cause of potentially serious image artifacts in the resulting CT images. These are commonly referred to as beam-hardening artifacts or in extreme cases as photon starvation and image saturation due to excessive beam attenuation (i.e. from iodine). Algorithmic corrections are available. These at best are approximate solutions, whereas attacking the root cause, too much iodine, is the preferred approach. Subsequently, it has now surprisingly been found that CT radiation dose reduction means, such as utilizing reduced x-ray tube voltages, should be accompanied with reduced iodine concentration in order to preserve artifact-free image quality.

In addition to reducing the radiation dose by lowering the tube voltage, other options are available. Any technique, including CT technology, hardware and algorithms, for reducing the X-ray radiation dose, combined with the administration of ultra-low concentrations of a contrast agent, is encompassed by the method of the invention. CT equipments settings, i.e. exposure parameters such as x-ray tube current, slice thickness, pitch or table speed can be adjusted to reduce the radiation dose. CT technology including axial scanning may be used. In such technique there is no overlap of slices, without significant decrease in speed. Further, tube current (mA or milliamperage) modulation may be performed, i.e. turning down the X-ray tube current when not needed, and in particular turning it down through thinner sections of the body. Milliamperage represents a second control of the output of the X-ray tube. This control determines how much current is allowed through the filament on the cathode side of the tube. If more current (and heating) is allowed to pass through the filament more electrons will be available in the “space charge” for acceleration to the x-ray tube target and this will result in a greater flux of photons when the high voltage circuit is energised. Similar approaches using kVp modulation based on patient size are also envisaged as an additional method for infant, child or adult patient radiation dose reduction.

In addition, a Garnet-based ceramic scintillator detector, which has a high temporal resolution, may be used. Such detectors provide more contrast from the same radiation dose. Further, such fast detectors can also accommodate dual-energy GSI (Gemstone Spectral Imaging) imaging from a single source (X-ray tube) by rapid kVp switching. Scanning with such Dual Energy CT (DECT) and using GSI processing, enables to obtain spectral information and the reconstruction of synthetic monochromatic images, such as between 40 and 140 keV. In one embodiment, the examination step of the method of the invention includes the use of DECT. Higher contrast is provided when using lower energy monochromatic DECT images, but due to reduced photon intensity such technique may suffer from higher noise levels. Software that improves image quality may further be used to suppress noise. Filtered back projection (FBP) and Adaptive Statistical Iterative Reconstruction (ASiR™), a reconstruction method that selectively sweeps noise from CT images, allow the radiation dose to be reduced with no change in spatial or temporal resolution.

Likewise: Iterative Reconstruction in Image Space (IRIS™), IDOSE and Quantum Noise Filter reduce image noise without loss of image quality or detail visualization. More complex iterative techniques, such as model-based iterative reconstruction (MBIR), such as Veo™, may lead to further noise and dose reductions or better image quality. Hence, in a further embodiment, the examination step of the method of the invention includes operating the equipment such that scanning with DECT, optionally combined with noise suppression, is performed. Such noise suppression is preferably selected from ASiR and MBIR. Combining DECT with noise suppression, improved contrast to noise is achieved. Further, using DECT, with or without additional dedicated noise suppression methods, allows for the use of an X-ray diagnostic composition with a significantly reduced iodine concentration. For instance, scanning with DECT, e.g. at radiation doses of 21.8 mGy and 12.9 mGy, showed that a reduction of about 25% in the concentration of iodine, compared to standard 120 kV scans, is allowed for (Example 6). Using DECT and noise suppression the usable energy window is increased without compromising on image quality.

With any such technique for reducing noise, the radiation dose can be reduced and together with reduced iodine concentration (i.e. ULC) adult, child or infant patient safety is further enhanced. In a preferred embodiment, the method of the invention includes a step of noise reduction, preferably through advanced image reconstruction and/or image filtration methods. Such noise reduction is achieved by selecting and operating available software, and it is preferably selected from ASiR and MBIR (Veo™). Compared to standard Filter Back Projection, both ASiR and MBIR significantly improve the contrast to noise radio, also in studies with iodine contrast. In a preferred embodiment, MBIR (Veo™) is used in the method of the invention.

The radiation dose needed is dependent on the procedure, on the region of interest, and on the weight, and age, of the patient. Hence, in a preferred embodiment, the invention provides a method of X-ray examination comprising administration to a body an X-ray diagnostic composition having an ultra-low concentration of iodine, applying a reduced kVp and limited mAs (milliampere×sec exposure level) for reduced X-ray radiation dose, and examining the body with a diagnostic device and compiling data from the examination, wherein the method further includes a step of noise reduction through advanced image reconstruction means.

With the method of the invention the radiation dose of a standard CT of abdominal region may be reduced by up to 50% from an average of 8 mSv (milliSevert) or less, of CT of central nervous system (spine) by up to 50% from an average of 5 mSv, and CT of chest by up to 50% from an average of 7 mSv. With the method of the invention, using an X-ray diagnostic composition with an ultra-low concentration of iodine and advanced reconstruction software, the radiation dose can, depending on the type of reconstruction, be reduced by 10%, 20%, 30%, 40% or even 50%, 60%, 70% or even 80%-90% compared to standard radiation doses, without compromising on the imaging quality.

As reported by Flicek the radiation dose during CTC can be reduced with 50% when ASIR is used, and the standard dose settings of 50 mAs is reduced to 25 mAs. With the method of the invention, using ultra-low concentration of iodine, the dose settings can be reduced similarly, i.e. from standard 50 mAs to e.g. 25 mAs.

In the method of the invention the X-ray contrast agent of the X-ray composition administered is any biocompatible X-ray attenuating agent with high atomic number. Preferably the X-ray contrast agents is an iodinated X-ray compound, preferably a non-ionic iodinated monomeric compound or a non-ionic iodinated dimeric compound as outlined in the first aspect of the invention. In another embodiment, the X-ray contrast agent comprises nanoparticles of high atomic number materials, This includes elements of atomic number 53 or higher, including, but not limited to, iodine (I), gadolinium (Gd), tungsten (VV), tantalum (Ta), hafnium (Hf), bismuth (Bi), gold (Au) and combinations thereof. The particles may be coated to improve elimination from the body and reduce toxicity. In the embodiment wherein the administered composition comprises an iodinated X-ray contrast agent together with a pharmaceutically acceptable carrier or excipient, the composition has an ultra-low concentration of iodine, as provided in the first aspect. If the contrast agent comprises nanoparticle materials the composition should include similar concentrations providing similar attenuation as iodine to X-rays. Preferably, the administered concentration of nanoparticles is in the range of 50-200 mg/kg body weight when administered.

In a preferred embodiment, the invention provides a method of X-ray examination comprising administration to a body an X-ray composition comprising an X-ray contrast agent with an ultra-low concentration of iodine, irradiating the body with a reduced radiation dose, e.g. by using a tube voltage lower than 150 kVp, such as 80 kVp, and tube currents in the 5-1000 mA range, such as in the 5-700 mA range, or in the 5-500 mA range, and examining the body with a diagnostic device, and compiling data from the examination.

Optionally, but preferably the examining of the body with a diagnostic device includes reconstructing the image using any reconstruction software and compiling data from the examination, using any image/data management system.

With the method of the invention it has been found that the image quality is at least maintained, good, or even improved compared to procedures wherein standard doses of radiation and standard concentrations of contrast agent are applied. Hence, by the methods and compositions of the invention the contrast to noise ratio is maintained, compared to standard methods and compositions, or even improved, to preserve or improve image quality. The CT attenuation value of iodinated enhancement is increased at a lower tube voltage, resulting in higher enhancement and/or maintained or better definition. The image quality, measured in Hounsfield Units (HU), obtainable by the method of the invention is typically 60-350 HU.

Image Quality (IQ) ranges for typical imaging procedures are e.g.:

Post Contrast Arterial Phase Density Measurements at regions of interests: Abdominal Aorta/Renal Artery/Kidney Cortex/Liver Parenchyma/Portal Vein/IVC=60-350 HU.
Post Contrast Venous Phase Density Measurements at various regions of interests: Abdominal Aorta/Renal Artery/Kidney Cortex/Liver Parenchyma/Portal Vein/IVC=80-350 HU.

The X-ray composition and the method of the invention may be used for the X-ray examination of different regions of interest, and for several types of indications. Examples are intra-arterial or intra venous administration of the X-ray composition for visualizing vascular structures, for visualising thoracic, abdominal neoplastic and non-neoplatic lesions, for indications related to head and neck, and for the evaluations of the periphery/body cavities.

In a third aspect the invention provides a method of X-ray examination comprising examining a body preadministered with an X-ray diagnostic composition as described in the first aspect, comprising the method steps of the second aspect of the invention. This aspect includes the same features and fall-backs as the two first aspects of the invention.

In a fourth aspect the invention provides an X-ray diagnostic composition comprising an iodinated X-ray contrast agent, wherein the composition has an ultra-low concentration of iodine, for use in a method of x-ray examination comprising administering the diagnostic composition to a body, applying a reduced X-ray radiation dose to the body, examining the body with a diagnostic device and compiling data from the examination. This aspect includes the same features and fall-backs as the two first aspects of the invention.

The methods of the invention may further include the steps of examining the body with a diagnostic device and compiling data from the examination and optionally analysing the data.

The invention is illustrated with reference to the following non-limiting examples and the accompanying drawings.

### BRIEF DESCRIPTION OF THE DRAWINGS

FIG. 1 shows the Impact of low kVp on attenuation at different concentration of iodine.

FIG. 2 shows the impact of low kVp computed tomographic (CT) on image attenuation without additional noise reduction methods, providing the contrast to noise at the centre of a phantom using the GE Gemstone detector and prep-based data processing and the Siemens Flash CT, at 80 and 120 kVp.

FIG. 3 shows the image quality (CNR) for the GE prep-based data system and the Siemens Flash CT when increasing the radiation from 80 to 140 kVp.

FIG. 4 shows the mass attenuation coefficient of Visipaque and other contrast media versus the radiation, keV.

FIG. 5 shows the image quality (CNR) versus the contrast media (Visipaque, named Vp) concentration.

FIG. 6 shows the normalized contrast to noise ratio (CNRD) measured in a phantom study for 80, 100 and 120 kVp scans using standard reconstruction and two types of iterative reconstruction methods, at standard and low radiation dose levels.

FIGS. 7-9 show in vivo minipig CT images acquired during the arterial phase after Visipaque administration. The solid arrow points to the aorta, the dashed arrow to muscle (quadratus lumborum).

FIGS. 10-12 show in vivo minipig CT images acquired during the venous phase after Visipaque administration. The solid arrow points to the liver.

### EXAMPLES

### Example 1

### The Impact of Low kVp Computed Tomographic (CT) on Contrast-to-Noise Ratio (CNR) without Special Noise Reduction Methods

Schindera et al (2008) Hypervascular Liver Tumors: Low Tube Voltage, High Tube Current Multi-Detector Row CT for Enhanced Detection—Phantom Study. Radiology (246): Number 1, January, 2008 evaluated the effect of a low tube voltage, high tube current computed tomographic (CT) technique on image noise, contrast-to-noise ratio (CNR), lesion conspicuity and radiation dose in simulated hypervascular liver lesions in a phantom.

This phantom containing four cavities (each of 3, 5, 8, and 15 mm in diameter) filled with various iodinated solutions to simulate hypervascular liver lesions, was scanned with a 64-section multi-detector row CT scanner at 140, 120, 100, and 80 kVp, with corresponding tube current-time product settings at 225, 275, 420, and 675 mAs, respectively. Results showed that radiation dose can be substantially reduced by using 80 kVp. Furthermore this kVp resulted in the highest CNR.
140 kVp; 225 mAs resulted in a radiation dose of 11.1 mSv.
120 kVp; 275 mAs resulted in a radiation dose of 8.7 mSv.
100 kVp; 420 mAs resulted in a radiation dose of 7.9 mSv.
80 kVp; 675 mAs resulted in a radiation dose of 4.8 mSv.

At a constant radiation dose, a reduction of tube voltage from 140 to 120, 100, and 80 kVp increased the iodine CNR by factors of at least 1.6, 2.4, and 3.6, respectively (p<0.001). At a constant CNR, corresponding reductions in Effective Dose ED (radiation dose) were by a factor of 2.5, 5.5, and 12.7, respectively (P<0.001). Thus equivalent or better conspicuity of iodinated structures is possible at 70% less radiation dose-sensitivity and specificity are equivalent, while dose is reduced from 18 mSv to SmSv.

Although the above results showed that using 80 kVp could substantially reduce that radiation dose, the image noise increased by 45% with the 80-kVp protocol compared with the 140-kVp protocol (p<0.001). This demonstrates that noise reduction through advanced image reconstruction methods is essential to image quality.

### Example 2

### The Impact of Low kVp Computed Tomographic (CT) on Image Attenuation without Special Noise Reduction Methods

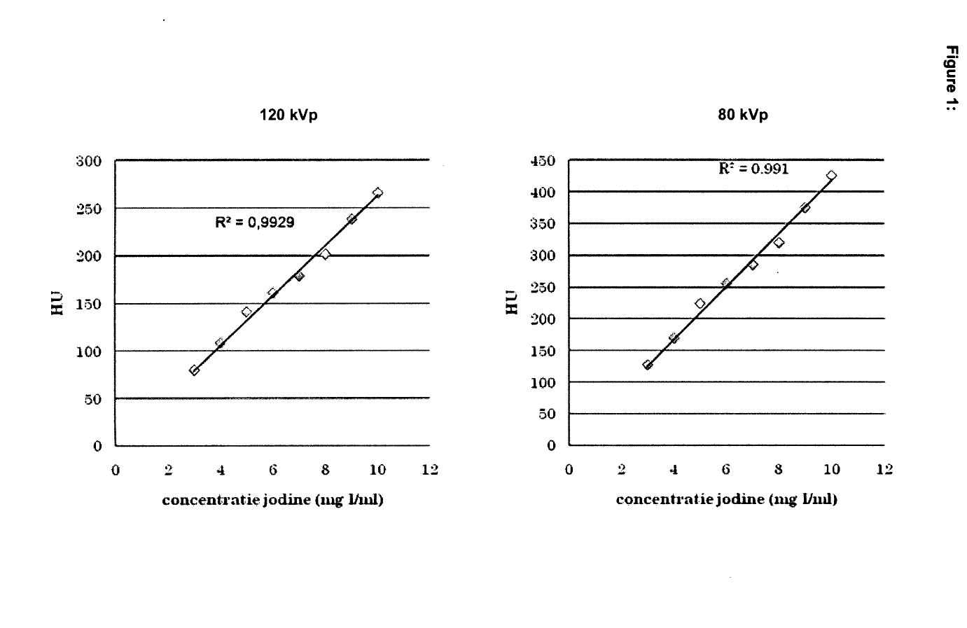

The inventors evaluated the effect of a low tube voltage on iodine CNR in a static phantom. The phantom contained cavities filled with various iodinated solutions (0-12 mgl/ml) to simulate filled blood vessels, and this was scanned with GE HD 750CT at 120 and 80 kVp. Results without adaptive statistical or model based reconstruction (ASiR/MBiR) showed that at 120 kVp ˜250 Hounsfield Unit (HU) attenuation was achieved with 9.5 mgl/ml iodinated contrast media, whereas under 80 kVp only 6 mgl/ml was needed for the same attenuation. This confirms Iodine HU values are greater at lower kVps because of the increasing attenuation coefficient of iodine at lower x-ray energies—see FIG. 1, which shows the Impact of low kVp on attenuation at different concentration of iodine. Such data suggest additional reconstruction with ASiR/MBiR will further enhance image conspicuity at low kVp, low iodine concentration and lower overall iodine dose in vivo. Results without special noise reduction methods showed higher attenuation at low kVp for all concentrations of iodine.

### Example 3

### Preservation of Low kVp Image Quality (IQ)

This examples shows that there is no need for high milliamperage (mA) when using low kVp to boost Image Quality. Special prep-based data processing boosts image fidelity and preserves low kVp Image Quality (IQ).

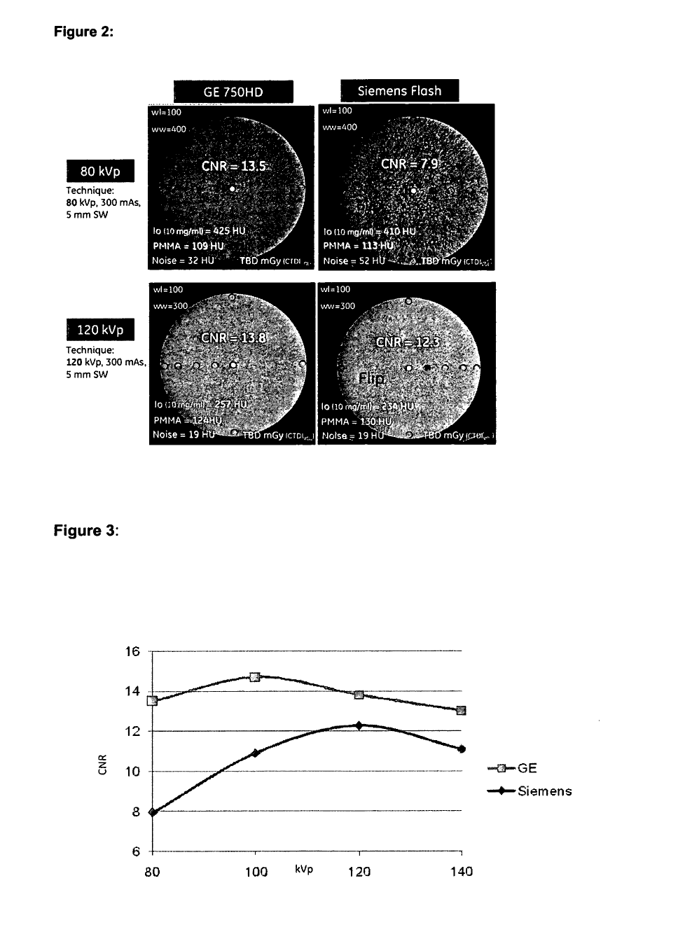

In an additional phantom study, a 32 cm poly-methyl methacrylate (PMMA) phantom was used with Iodine at 10 mg/ml and noise was measured at the centre of the phantom. In this study the GE HD 750 system using special prep-based data processing to improve low signal level performance and boost image fidelity and preserve low kVp image quality delivered the same image quality (IQ, CNR) at the same mAs at 80 kVp versus 100/120/140 kVp. Indeed using a GE HD 750 CT 80 kVp and 300 mAs yielded a contrast to noise ratio (CNR) of 13.5 compared to a contrast to noise ratio of 13.8 at 120 kVp and 300 mAs, showing CNR is maintained at lower kVp. Such data suggests that in Iodine contrast studies there is no need for high mA at 80 kVp, and 0-500 mA is sufficient. Other equipment without special prep-based data processing such as the Siemens Flash CT, 80 kVp and 300 mAs yielded CNR of 7.9 compared to a CNR of 12.3 at 120 kVp and 300 mAs. Improvement in soft tissue conspicuity at higher mA may be needed. FIG. 2 shows the impact of low kVp computed tomographic (CT) on image attenuation without additional noise reduction methods, providing the contrast to noise at the centre of a phantom using the GE Gemstone detector and prep-based data processing and the Siemens Flash CT, at 80 and 120 kVp. FIG. 3 shows the image quality (CNR) for the GE prep-based data system and the Siemens Flash CT when increasing the radiation from 80 to 140 kVp.

### Example 4

### Improvement in Dual-Energy Image Quality (IQ) in Phantoms when Contrast Media Rather than Elemental Iodine is Properly Modelled

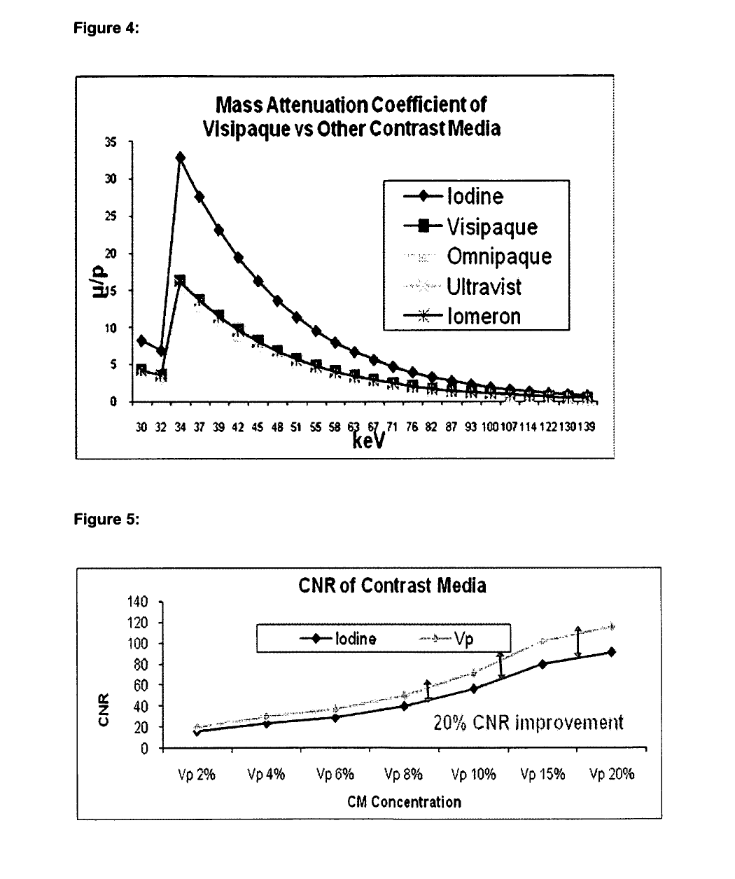

When tuning projection-based Basis Materials Decomposition in dual energy applications, such as elemental iodine, to the specific molecular structures of contrast media such as Visipaque, significant dual-energy image quality (IQ) improvements are exhibited. Elemental Iodine is only a rough approximation of today's complex contrast media (CM) chemistry, so image quality in phantoms improved when doing proper CM modelling. Proper elemental modelling of CM can improve “iodine” and “water” images, both in iodine CNR and in purity of water and contrast material separation. FIGS. 4 and 5 show that moving from elemental iodine to contrast media modelling, e.g. Visipaque, in projection-based Basis Materials Decomposition, optimizes image conspicuity. FIG. 4 shows the mass attenuation coefficient of Visipaque and other contrast media versus the radiation, keV. FIG. 5 shows the image quality (CNR) versus the contrast media (Visipaque, named Vp) concentration. A 10% Vp concentration hence means 10 grams of Visipaque 320 mgl/ml is added 90 grams of water. Such referencing to contrast media rather than to elemental iodine leads to a 20% increase in CNR in phantom tests and would further enhance the conspicuity of contrast media with an ultra-low concentration of iodine, thus enabling great patients safety benefits. Elemental analysis of contrast media, e.g. Visipaque, and iodine, reveals characteristic photoelectric and Compton effect attenuation coefficient behaviour and image-based Materials Decomposition (MD).

### Example 5

### Low kVp Computed Tomography (CT) and Iterative Reconstruction Techniques Enable Decreased Iodine Concentration with Equivalent Contrast-to-Noise Ratio (CNRD) as High kVp and High Iodine Concentration

The purpose of this study was to assess iodine contrast enhancement with 80 kVp and 100 kVp scans and two types of iterative reconstruction methods compared to a standard 120 kVp acquisition and reconstruction. Ten tubes with iodine contrast (Iodixanol 320 mgl/ml) concentrations diluted from 1 to 10 mgl/ml were inserted in a CT performance phantom (CIRS, Norfolk Va.). The phantom was scanned on a HD 750 CT scanner (GE Healthcare) with 120 kVp, 100 kVp and 80 kVp at standard and low radiation dose levels (CTDIvol (volume CT dose index) 10.7 and 2.7 mGy). Projection data was reconstructed with standard filtered back projection (FBP) and two types of iterative reconstruction; Adaptive Statistical Iterative Reconstruction (ASIR) and Model Based Iterative Reconstruction (MBIR) alternatively known as “Veo”. ASIR level was set at a clinically meaningful level, 60% (which accords with the standard of care in the hospital setting) and at 100%. Image quality was assessed by measuring the dose normalized contrast to noise ratio (CNRD) in the examined contrast tubes.
The CNRD remained linear (r2>0.99) as a function of iodine concentration at 120, 100 and 80 kVp acquisitions. See FIG. 6.
With standard FBP, CNRD increased for low 80 kVp acquisitions by 24% compared to 120 kVp.
For all three acquisitions—120, 100 and 80 kVp, CNRD increased by an average of 47% (range 44-50%) with ASIR (60%) iterative reconstruction compared to FBP. See FIG. 6.
There was no significant difference in the obtained CNRD between high and low radiation dose (CTDIvol) levels using ASIR.

In contrast to this, the results from Veo were clearly influenced by the radiation dose level:
At standard radiation dose level (10.8 mGy), CNRD increased by an average of 60% (range 56-64%) compared to FPB, whereas at low radiation dose level (2.7 mGy) CNRD increased by 103% (range 96-110%).
For equal CNRD, using 80 kVp allows a reduction of iodine concentration by about 29% compared to a standard 120 kVp scan.
With ASIR and Veo the allowed reduction of iodine concentration increased up to 53% and 61% respectively. At the low dose level, Veo allows a iodine concentration reduction of 68%.

Compared to standard FBP, both types of iterative reconstruction, ASIR and Veo, significantly improved CNRD in iodine contrast studies. The relative benefit of ASIR is independent of radiation dose. With Veo however, the relative CNRD increased for lower radiation doses. These results illustrate the potential to decrease iodine concentration and/or decrease patient radiation dose when applying iterative reconstruction on low kVp scans.

### Extrapolation to the Clinical Setting:

Since CNRD is equal at 80 kVp, this allows a reduction of iodine concentration by about 29% compared to a standard 120 kVp scan. These data suggest that, given a relationship between the concentration of injected iodinated contrast agent and the concentration appearing in blood vessels during clinical angiographic CT procedures, the injected (concentration in vial) concentration may be reduced from standard concentrations e.g. from 320 mgl/ml to 227.2 mgl/ml (i.e. 71% of 320 mgl/ml). It follows that, if the volumes of injected iodinated contrast agent remain the same and the concentration of iodine based contrast agent is reduced, the total amount of injected iodinated contrast agent into the body will be reduced. This reduction in overall amount of iodinated contrast agent would have fewer side effects (especially renal) for the patient and confer significant patient safety benefits.

Algorithmic reconstruction of these data with ASIR and Veo showed iodine concentration may be further reduced, by up to 53% and 61% respectively. These data indicate that vial concentration may be further reduced from standard concentrations (e.g. 320 mgl/ml) to 150.4 mgl/ml and 124.8 mgl/ml respectively through the use of iterative reconstruction methods. Furthermore, since at the low radiation dose level (2.7 mGy) model based iterative reconstruction using Veo implies iodine concentration may be reduced by 68%, this suggests vial concentrations may be further reduced to 102.4 mgl/ml. Thus it follows that, if the volumes of injected iodinated contrast agent remain the same and the concentration of iodine based contrast agent is even further reduced, the total amount of injected iodinated contrast agent into the body can be drastically lowered with Veo, such as to a concentration below 100 mgl/ml. This additional reduction in overall amount of iodinated contrast agent would lead to even fewer side effects for the patient and confer significant patient safety benefits, especially those subjects who would be susceptible potential adverse events such as to iodinated contrast agent-induced renal dysfunction or contrast media induced acute kidney injury.

### Example 6

### Dual Energy Computed Tomography (DECT) and Iterative Reconstruction Techniques Enable Decreased Iodine Concentration with Improved Contrast-to-Noise Ratio (CNR)

Scanning with Dual Energy CT (DECT) and the use of Gemstone Spectral Imaging (GSI) processing enables spectral information to be obtained by reconstructing synthetic monochromatic images between 40 and 140 keV. Images from low energy selections (<70 keV) typically result in higher contrast enhancement but suffer from high noise levels due to reduced photon intensity. Since these noise levels can be reduced by introducing iterative reconstruction, the purpose of this study was to compare iodine contrast enhancement with two types of DECT, one with and one without advanced noise suppression.

Ten tubes containing iodinated contrast agent (Visipaque (Iodixanol) 320 mgl/ml) diluted to concentrations found in blood vessels after the administration of iodinated contrast media (1 to 10 mgl/ml) were inserted in a CT performance phantom (CIRS, Norfolk Va.). The phantom was scanned at two radiation doses (CTDIvol (volume CT dose index) 21.8 mGy and 12.9 mGy) on a HD 750 CT scanner (GE Healthcare) with standard 120 kVp and by DECT with and without advanced noise suppression. Monochromatic images were retrieved by the GSI spectral viewer. Image quality was evaluated by assessing the contrast to noise ratio (CNR) as a function of keV selection.

The CNR remained linear (r2>0.99) as a function of iodinated contrast agent concentration for all of the investigated acquisition protocols. For all iodine concentrations tested, both DECT scans show an improved maximum CNR close to 36% compared to the standard 120 kVp scan at the same radiation dose (21.8 mGy).

Without advanced noise suppression, a maximum CNR peak was observed at 68 keV with a rapid drop at lower energies due to the domination of noise. This CNR drop is prevented with advanced noise suppression such that the CNR remains preserved in a larger energy window (40-70 keV). At both radiation dose levels, both GSI versions (with and without noise suppression) allow a reduction of iodinated contrast agent concentration by about 25% compared to a standard 120 kVp scan for equal CNR. This phantom study shows that iodine CNR can be drastically improved by using DECT and that adding advanced noise suppression increases the usable energy window without compromising image quality. The results illustrate the potential to either decrease iodine concentration and/or decrease patient radiation dose when applying iterative reconstruction on DECT.

### Extrapolation to the Clinical Setting:

GSI versions allow a reduction of iodinated contrast agent concentration by about 25% compared to a standard 120 kVp scan for equal CNR. These data suggest that, given a relationship between the concentration of injected iodinated contrast agent and the concentration appearing in blood vessels during clinical angiographic CT procedures, the injected (concentration in vial) concentration may be reduced from standard concentrations e.g. 320 mgl/ml to 240 mgl/ml. It follows that, if the volumes of injected iodinated contrast agent remain the same and the concentration of iodine based contrast agent is reduced, the total amount of injected iodinated contrast agent into the body will be reduced. This reduction in overall amount of iodinated contrast agent would have fewer side effects (especially renal) for the patient and confer significant patient safety benefits.

### Example 7

### The Combination of Decreased Iodine Concentration, Decreased Radiation Dose and Advanced Reconstruction Techniques Maintains the Signal-to-Noise Ratio (SNR) of Abdominal Contrast Enhanced CT Images in the Pig

An anaesthetized minipig (abdominal maximum and minimum diameters 36 cm and 20 cm, respectively) was imaged 3 times (imaging protocols 1, 2 and 3, Tables 3 and 4) on a Discovery CT 750 HD. Visipaque (60 mL) was injected at a rate of 2 mL/s into a jugular vein, followed by a 20 mL saline flush at the same injection rate. There was at least a 2 day washout period between each scanning session.

Protocol 1 with a Visipaque concentration of 320 mg l/mL and 120 kVp tube voltage represents current standard of care (SoC) imaging for humans. Automated tube current modulation (≦500 mA) was used with a noise index level of 30 and a tube rotation time of 0.7 s. Post-contrast CT images were acquired during the arterial phase, the portal venous phase, the venous phase and the late phase. Image reconstruction was done by (1) FBP, (2) ASiR 60% and (3) Veo. Pixel size was 0.703 mm×0.703 mm×0.625 mm.

Iodine contrast enhancement was assessed by measuring the signal-to-noise ratio (SNR) of circular regions of interest (ROI), see Tables 3 and 4. The SNR is calculated as ratio of mean ROI intensity in HU and standard deviation (SD). ROIs were placed in aorta and muscle (quadratus lumborum) in arterial phase images and in liver in venous phase images.

TABLE 3
Image acquisition and analysis data of arterial phase images
covering aorta and muscle. CTDIvol: volume CT dose index

| | Visipaque | Tube | | | | | |
|---|---|---|---|---|---|---|---|
| Proto- | concen- | volt- | Dose | | ASiR | | |
| col | tration | age | CTDIᵥₒₗ | FBP | 60% | Veo | |
| number | [mg l/ml] | [kVp] | [mGy] | [SNR] | [SNR] | [SNR] | Fig. |
| 1 | 320 | 120 | 6.7 | 8.3 | | | 7 |
| 2 | 170 | 80 | 3.2 | | 7.4 | 12.8 | 8A, |
| | | | | | | | 9A |
| 3 | 120 | 80 | 6.4 | | 8.5 | 14.2 | 8B, |
| | | | | | | | 9B |

TABLE 4
Image acquisition and analysis data of venous phase
images covering liver. CTDIvol: volume CT dose index

| | Visipaque | Tube | | | | | |
|---|---|---|---|---|---|---|---|
| Proto- | concen- | volt- | Dose | | ASiR | | |
| col | tration | age | CTDIᵥₒₗ | FBP | 60% | Veo | |
| number | [mg l/ml] | [kVp] | [mGy] | [SNR] | [SNR] | [SNR] | Fig. |
| 1 | 320 | 120 | 6.7 | 4.3 | | | 10 |
| 2 | 170 | 80 | 3.2 | | 3.5 | 8.1 | 11A, |
| | | | | | | | 12A |
| 3 | 120 | 80 | 6.4 | | 4.7 | 8.1 | 11B, |
| | | | | | | | 12B |

The same SNR (within 15%) is observed with protocol 1 & FBP reconstruction, protocol 2 & ASIR 60% reconstruction, and protocol 3 & ASIR 60% reconstruction. The SNR with protocols 2 and 3 and Veo reconstruction is approximately twice as large.

### CONCLUSIONS

a similar image quality in terms of SNR is observed with a reduced tube current of 80 kVp (compared to SoC setting of 120 kVp) and ASiR 60% (compared to standard SoC FBP method) when at the same time (a) reducing iodine contrast concentration to 170 mg l/mL and halving the radiation dose, or (b) reducing iodine contrast concentration further to 120 mg l/mL and keeping the radiation dose at the same level as in the SoC setting.

### Extrapolation to the Clinical Setting:

These data unexpectedly demonstrate that SNR is similar in the arterial phase i.e. 7.4 and 8.5 when the iodine concentration is reduced to 170 mgl/ml and 120 mgl/ml i.e. ˜47% and −62% lower than 320 mgl/ml when data are reconstructed using ASIR. Even more surprisingly, SNR is even higher in the arterial phase i.e. 12.8 and 14.2 when data are reconstructed using Veo. Similarly, in the venous phase SNR is similar i.e. 3.5 and 4.7 when the iodine is reduced to 170 mgl/ml and 120 mgl/ml i.e. ˜47% and ˜62% lower than 320 mgl/ml when data are reconstructed using ASIR. Once again, and surprisingly SNR is even higher in the venous phase i.e. 8.1 and 8.1 when data are reconstructed using Veo.

These data suggest that, given a relationship between the concentration of injected iodinated contrast agent and the concentration appearing in blood vessels during clinical angiographic CT procedures, the injected (concentration in vial) concentration may be reduced from standard concentrations e.g. from 320 mgl/ml to between 170 mgl/ml and 120 mgl/ml. It follows that, if the volumes of injected iodinated contrast agent remain the same and the concentration of iodine based contrast agent is reduced, the total amount of injected iodinated contrast agent into the body will be reduced. This reduction in overall amount of iodinated contrast agent would have fewer side effects for the infant, child and adult patient and confer significant patient safety benefits, especially those subjects with immature kidneys, or those who would be susceptible potential adverse events such as to iodinated contrast agent-induced renal dysfunction or contrast media induced acute kidney injury.

Furthermore, the respective reduction in radiation dose levels to 6.4 and 3.2 mGy after 120 mgl/ml/80 kVp and 170 mgl/ml/80 kVp compared to 6.7 mGy (320 mgl/ml and 120 kVp) also suggest lower radiation levels are simultaneously possible. Since exposure to radiation at an early age carries a risk to organs and tissues a lower radiation exposure would be of considerable additional benefit in these subjects.

### Figure Captions:

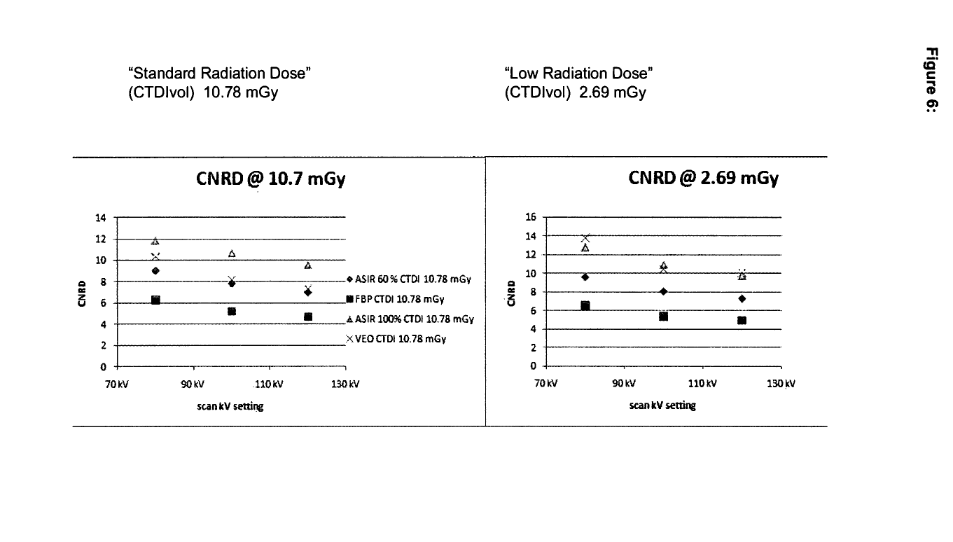

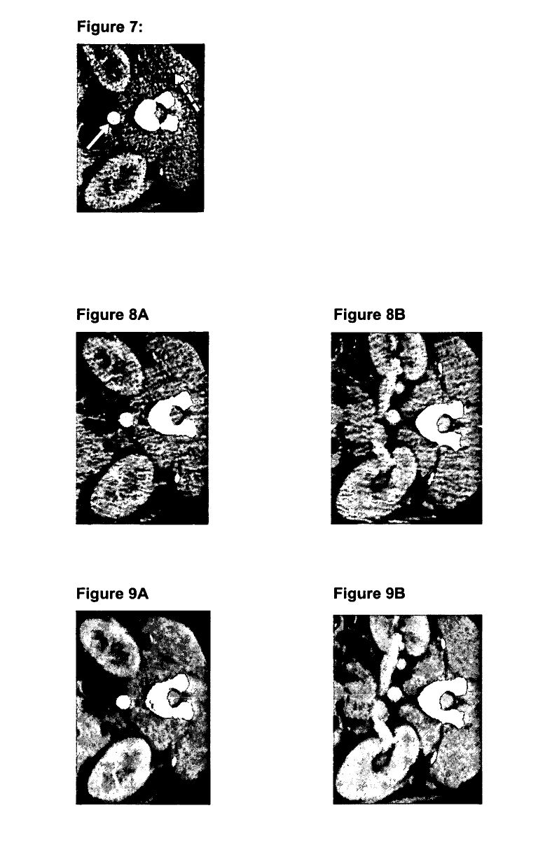

FIGS. 7-9: In vivo minipig CT images acquired during the arterial phase after Visipaque administration. The solid arrow points to the aorta, the dashed arrow to muscle (quadratus lumborum). Corresponding CT settings are listed in Table 3. Reconstruction was done with FBP (FIG. 7), ASiR 60% (FIGS. 8A, 8B), and Veo (FIGS. 9A, 9B).

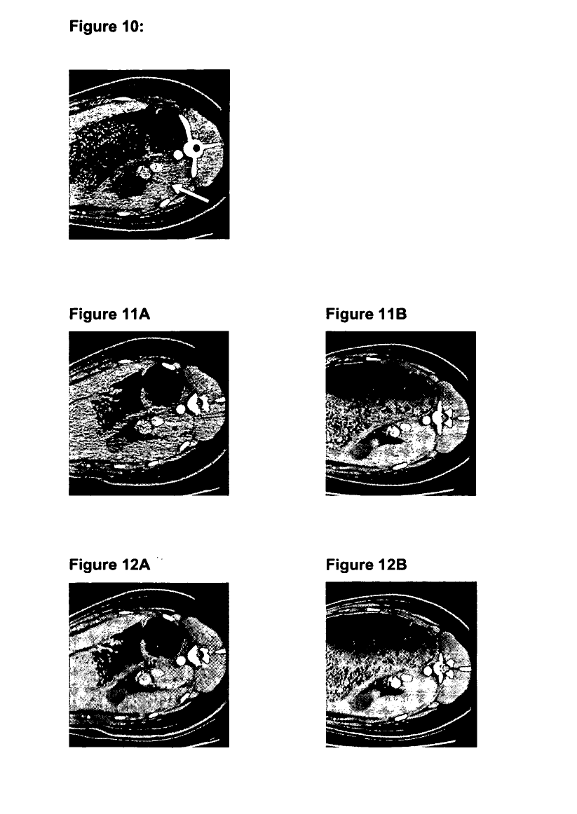

FIGS. 10-12: In vivo minipig CT images acquired during the venous phase after Visipaque administration. The solid arrow points to the liver. Corresponding CT settings are listed in Table 4. Reconstruction was done with FBP (FIG. 10), ASiR 60% (FIGS. 11A, 11B), and Veo (FIGS. 12A, 12B).

What is claimed is:

1. A composition comprising an iodinated X-ray contrast agent and a pharmaceutically acceptable carrier or excipient, wherein the composition has a concentration of iodine of less than 100 mg I/ml.

2. (canceled)

3. A composition as claimed in claim 1 wherein said X-ray contrast agent is a non-ionic iodinated monomeric, dimeric, trimeric, tetrameric or pentameric compound.

4. A composition as claimed in claim 3 wherein said X-ray contrast agent is iodixanol or the compound of formula II:

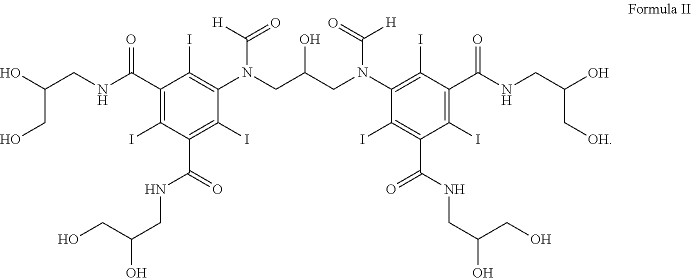 [SMILES: O=CN(CC(O)CN(C=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I]

5. (canceled)

6. A method comprising
administering to a body a composition comprising an X-ray contrast agent and a pharmaceutically acceptable carrier or excipient, wherein said composition has a concentration of iodine of 10-170 mg I/ml,
applying an X-ray radiation dose to the body wherein said radiation dose is provided by a tube voltage energy in the range of 70-140 kVp,
examining said body with a diagnostic device and compiling data from said examining step.

7. (canceled)

8. A method as claimed in claim 6 wherein said composition has a concentration of iodine of less than 150 mg I/ml.

9. A method as claimed in claim 8, wherein said contrast agent is iodinated and, wherein said dose of radiation has an average energy spectrum substantially corresponding to the k-edge of iodine.

10. (canceled)

11. A method as claimed in claim 6 wherein said reduced X-ray radiation dose is further provided by a tube current in the range of 5-1000 mA.

12. A method as claimed in claim 6 wherein said radiation dose is >30% lower than standard radiation doses.

13. A method as claimed in claim 6 further comprising a step of noise reduction through an advanced image reconstruction method.

14. A method as claimed in claim 13 wherein said noise reduction is selected from the iterative image reconstruction methods ASiR and MBIR.

15. A method as claimed in claim 6 wherein said reduced X-ray radiation dose is further provided by Dual Energy CT.

16. A method as claimed in claim 6 wherein the volume of said composition is between 1 and 50 ml.

17. A method as claimed in claim 6 wherein said X-ray contrast agent comprises nanoparticles of a high atomic number.

18. (canceled)

19. (canceled)

20. A method as claimed in claim 6 wherein said composition has a concentration of iodine of less than 100 mg I/ml.
